(19) United States
(12) Patent Application Publication
(10) Pub. No.: US 2023/0191137 A1
(43) Pub. Date: Jun. 22, 2023

(54) IMPLANTABLE MEDICAL DEVICE AND METHOD OF FORMING SAME

(71) Applicant: Medtronic, Inc., Minneapolis, MN (US)

(72) Inventors: John Mertz, Lino Lakes, MN (US); Kamal Deep Mothilal, Maple Grove, MN (US); Suping Lyu, Maple Grove, MN (US)

(21) Appl. No.: 17/866,734

(22) Filed: Jul. 18, 2022

Related U.S. Application Data
(60) Provisional application No. 63/291,670, filed on Dec. 20, 2021.

(51) Int. Cl. A61N 1/375; C08L 75/04

(52) U.S. Cl. CPC A61N 1/3752; C08L 75/04

(57) ABSTRACT

Various embodiments of an implantable medical device and a method of forming such device are disclosed. The device includes a housing that has a first portion and a second portion connected to the first portion. The first portion includes a polymer material and the second portion includes a transparent polymer material. The housing further includes a lead bore that extends between a first end at an outer surface of the housing and a second end disposed within the housing.

### CROSS-REFERENCE TO RELATED APPLICATION

The present application claims the benefit of U.S. Provisional Application No. 63/291,670, filed Dec. 20, 2021, which is incorporated herein by reference in its entirety.

### TECHNICAL FIELD

This disclosure generally relates to an implantable medical device.

### BACKGROUND

Implantable medical device systems such as implantable pacemakers can deliver pacing pulses to a patient’s heart and monitor conditions of the patient’s heart. In some examples, the implantable pacemaker includes an electronic assembly disposed within a housing and one or more electrical leads. The electronic assembly may, for example, be implanted in a small pocket in the patient’s chest. The electrical leads can be coupled to the electronic assembly, which may include one or more electronic components that generate pacing pulses and/or sense cardiac electrical activity. The electrical leads may extend from the electronic assembly to a target site (e.g., an atrium and/or a ventricle) such that electrodes disposed on or within the electrical leads are positioned at the target site. The electronic assembly may provide electrical stimulation to the target site and/or monitor cardiac electrical activity at the target site via the electrodes.

A pacemaker typically has a hermetically sealed metal housing that encloses circuitry and a power source in a moisture free environment. A polymeric connector module can be connected to the metal housing, where the connector module provides a lead bore for insertion of a lead and one or more electrical contacts that are configured to electrically connect the inserted lead to circuitry disposed within the housing. The connector module can also include a setscrew block that is adapted to retain the lead within the lead bore.

### SUMMARY

The techniques of this disclosure generally relate to an implantable medical device and system that includes such device. The implantable medical device can include a housing and an electronics module disposed within the housing. The housing can include a first portion and a second portion that are connected using any suitable technique. The first portion of the housing can include a polymer material such as at least one of a thermoplastic polymer material (e.g., PEEK or a liquid crystal polymer) or a thermoset polymer material (e.g., an epoxy). Further, the second portion of the housing can include a transparent polymer.

This disclosure includes without limitation the following clauses:

Clause 1: An implantable medical device that includes a housing that has a first portion and a second portion connected to the first portion. The first portion includes a polymer material and the second portion includes a transparent polymer material. The housing further includes a lead bore that extends between a first end at an outer surface of the housing and a second end disposed within the housing.

Clause 2: The device of Clause 1, where the polymer material of the first portion of the housing includes at least one of a thermoplastic material, a thermoset material, a polymer composite, or a polymer blend.

Clause 3: The device of Clause 2, where the polymer material of the first portion of the housing includes a thermoplastic polymer material including at least one of liquid-crystal polymer (LCP), polyetherether ketone (PEEK), polyether kotone (PEK), polyoxidemethylene (POM), polyimide, polypropylene, polyurea, polyester, polyphenylene oxide, polyamide, polytetrafluorethylene, polysulfone, polycarbonate, polystyrene or its copolymer, polyacrylate, polymethylacrylate, cellulose derivative, epoxy, or phenolic resin.

Clause 4: The device of Clause 3, where the thermoplastic polymer material includes at least one of PEEK or LCP.

Clause 5: The device of any one of Clauses 1-4, where the polymer material of the first portion of the housing includes a crystallinity of at least 1%.

Clause 6: The device of any one of Clauses 1-5, where the polymer material of the first portion of the housing includes a Tₘ of at least 50° C.

Clause 7: The device of any one of Clauses 1-6, where the polymer material of the first portion of the housing includes a water solubility of no greater than 5%.

Clause 8: The device of any one of Clauses 1-7, where the transparent polymer material includes polyurethane.

Clause 9: The device of any one of Clauses 1-8, further including an electronics module disposed within the housing and including a power source and circuitry electrically connected to the power source.

Clause 10: The device of Clause 9, where the lead bore is adapted to connect an external lead to the circuitry of the electronics module.

Clause 11: The device of Clause 10, further including a conductor that electrically connects the circuitry disposed within the housing to a contact disposed within the lead bore, where the contact is adapted to be electrically connected to a lead contact of the lead.

Clause 12: The device of any one of Clauses 1-11, where the first portion of the housing encloses the second portion of the housing.

Clause 13: The device of Clause 12, where the second portion forms a window disposed within the first portion of the housing.

Clause 14: The device of any one of Clauses 1-13, further including a coating disposed over at least a portion of an outer surface of the housing.

Clause 15: The device of Clause 14, where the coating includes a ceramic coating.

Clause 16: The device of Clause 15, where the ceramic coating includes at least one of aluminum oxide, glass, mica, titanium oxide, titanium nitride, vanadium oxide, niobium oxide, zirconium oxide, hafnium oxide, silicon oxide, or silicon nitride.

Clause 17: The device of Clause 14, where the coating includes a multilayer coating.

Clause 18: The device of Clause 17, where the multilayer polymer coating includes at least one of epoxy, parylene, polyimide, silicone, acrylic, or vinyl.

Clause 19: The device of any one of Clauses 1-18, further including a desiccant disposed within the housing including at least one of molecular sieve, calcium sulfate, silica gel, lithium sulfate, or polyanhydride.

Clause 20: The device of any one of Clauses 1-19, where the first portion of the housing is molded to the second portion of the housing.

Clause 21: The device of any one of Clauses 1-19, where the first portion of the housing is adhered to the second portion of the housing.

Clause 22: The device of any one of Clauses 1-19, where the first portion of the housing is mechanically fastened to the second portion of the housing.

Clause 23: The device of any one of Clauses 1-22, where the housing further includes at least one tab or slot disposed on a mating surface of the first portion of the housing and that is adapted to receive a respective tab or slot disposed on a mating surface of the second portion of the housing.

Clause 24: An implantable medical device system that includes an implantable medical device and a lead. The implantable medical device includes a housing that has a first portion and a second portion connected to the first portion. The first portion includes a polymer material and the second portion including a transparent polymer material. The housing further includes a lead bore that extends between a first end at an outer surface of the housing and a second end disposed within the housing. The implantable medical device further includes an electronics module disposed in the housing and including a power source and circuitry electrically connected to the power source. At least a portion of the lead is adapted to be disposed within the lead bore and electrically connected to the circuitry of the electronics module.

Clause 25: The system of Clause 24, where the lead bore of the implantable medical device includes a contact that is electrically connected to the circuitry of the electronics module by a conductor, where a lead contact of the lead is adapted to be electrically connected to the contact of the lead bore when the at least a portion of the lead is disposed within the lead bore.

Clause 26: The system of any one of Clauses 24-25, where the implantable medical device is an implantable defibrillator.

Clause 27: The system of any one of Clauses 24-25, where the implantable medical device is a neurostimulator.

Clause 28: The system of any one of Clauses 24-27, where the polymer material of the first portion of the housing of the implantable medical device includes at least one of a thermoplastic polymer material, a thermoset polymer material, a polymer composite, or a polymer blend.

Clause 29: The system of Clause 28, where the polymer material of the first portion of the housing of the implantable medical device includes PEEK.

Clause 30: The system of Clause 28, where the polymer material of the first portion of the housing of the implantable medical device includes LCP.

Clause 31: The system of any one of Clauses 24-30, where the polymer material of the first portion of the housing of the implantable medical device includes a crystallinity of at least 1%.

Clause 32: The system of any one of Clauses 24-31, where the polymer material of the first portion of the housing of the implantable medical device includes a Tₘ of at least 50° C.

Clause 33: The system of any one of Clauses 24-32, where the polymer material of the first portion of the housing of the implantable medical device includes a water solubility of no greater than 5%.

Clause 34: The system of any one of Clauses 24-33, where the transparent polymer material of the housing of the implantable medical device includes polyurethane.

Clause 35: The system of any one of Clauses 24-34, further including a ceramic coating disposed over at least a portion of an outer surface of the housing of the implantable medical device.

Clause 36: The system of Clause 35, where the ceramic coating includes at least one of aluminum oxide, glass, mica, titanium oxide, titanium nitride, vanadium oxide, niobium oxide, zirconium oxide, hafnium oxide, silicon oxide, or silicon nitride.

Clause 37: The system of any one of Clauses 24-36, where the first portion of the housing encloses the second portion of the housing.

Clause 38: The system of Clause 37, where the second portion of the housing forms a window disposed within the first portion of the housing.

Clause 39: The system of any one of Clauses 24-38, further including a multilayer coating disposed over at least a portion of an outer surface of the housing.

Clause 40: The system of Clause 39, where the multilayer coating includes at least one of epoxy, parylene, polyimide, silicone, acrylic, or vinyl.

Clause 41: The system of any one of Clauses 24-40, further including a desiccant disposed within the housing of the implantable medical device.

Clause 42: The system of Clause 41, where the desiccant includes at least one of molecular sieve, calcium sulfate, silica gel, lithium sulfate, or polyanhydride.

Clause 43: The system of any one of Clauses 24-42, where the first portion of the housing is molded to the second portion of the housing.

Clause 44: The system of any one of Clauses 24-42, where the first portion of the housing is adhered to the second portion of the housing.

Clause 45: The system of any one of Clauses 24-42, where the first portion of the housing is mechanically fastened to the second portion of the housing.

Clause 46: The system of any one of Clauses 24-45, where the first portion of the housing further includes at least one tab or slot disposed on a mating surface of the first portion of the housing that is adapted to receive a respective tab or slot disposed on a mating surface of the second portion of the housing.

Clause 47: A method of forming an implantable medical device system. The method includes forming a first portion of a housing that has a polymer material; forming a second portion of the housing that includes a transparent polymer material; forming a lead bore in the second portion of the housing that extends between a first end at an outer surface of the housing and a second end disposed within the housing; and connecting the first portion of the housing to the second portion of the housing.

Clause 48: The method of Clause 47, where connecting the first portion of the housing to the second portion of the housing includes molding the second portion onto the first portion.

Clause 49: The method of Clause 48, where molding the second portion of the housing onto the first portion of the housing includes overmolding the second portion onto the first portion.

Clause 50: The method of any one of Clauses 47-49, where forming the first portion of the housing includes forming at least one of a tab or slot on a mating surface of the housing.

Clause 51: The method of Clause 50, where forming the second portion of the housing includes forming at least one of a tab or slot on a mating surface of the second portion, where the at least one tab or slot of the first portion is adapted to mate with the respective at least one tab or slot of the second portion when the first portion is connected to the second portion.

Clause 52: The method of any one of Clauses 47-51, further including inserting at least a portion of a lead into the lead bore such that a lead contact of the lead is electrically connected to circuitry of an electronics module disposed within the housing of the implantable medical device.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a schematic view of an implantable medical device system disposed within a body of a patient.

FIG. 2 is a schematic perspective view of the implantable medical device system of FIG. 1.

FIG. 3 is a schematic plan view of an implantable medical device of the system of FIG. 2.

FIG. 4 is a schematic plan view of the implantable medical device of FIG. 3 with a portion of a housing of the device removed for clarity.

FIG. 5 is a schematic cross-section view of the implantable medical device of FIG. 3.

FIG. 6 is a schematic cross-section view of the implantable medical device system of FIG. 2.

FIG. 7 is a flowchart of one embodiment of a method of forming the implantable medical device of FIG. 2.

### DETAILED DESCRIPTION

The techniques of this disclosure generally relate to an implantable medical device and system that includes such device. The implantable medical device can include a housing and an electronics module disposed within the housing. The housing can include a first portion and a second portion that are connected using any suitable technique. The first portion of the housing can include a polymer material such as at least one of a thermoplastic polymer material (e.g., PEEK or a liquid crystal polymer) or a thermoset polymer material (e.g., an epoxy). Further, the second portion of the housing can include a transparent polymer.

Implantable medical devices such as pacemakers can be disposed within a body of patient and provide various types of treatments to the patient through delivery of electric or other types of signals. Because it is disposed within the body, these devices are exposed to fluids or tissue that may be directed into a housing of the device and potentially damage or contaminate electronics or power sources disposed within the housing.

To prevent ingress of these fluids, the housing can be hermetically sealed; however, certain types of materials such as titanium or other metals may be required to form such hermetically sealed housings. These materials can be expensive, and techniques that can be utilized to seal these materials together can also be expensive and present various technical challenges. Further, in circumstances where an implantable medical device may be for short term use, expensive materials such as titanium may not be desirable.

One or more embodiments of an implantable medical device described herein may include a housing that includes one or more polymeric materials that may be less expensive than materials that are typically used for such devices. For example, an implantable medical device can include a housing having a first portion that is connected to a second portion. The first portion can include a polymer such as PEEK or liquid crystal polymer, and the second portion can include a transparent polymer such as polyurethane. The polymer material of the first portion can provide a moisture barrier to prevent ingress of contaminants into an interior of the housing and potentially affecting performance of a power source or circuitry disposed within the housing. Further, the transparent polymer of the second portion can allow a clinician to view a lead bore that is disposed in the second portion as a portion of a lead is disposed within the lead bore. In particular, the transparent portion can allow the clinician to determine that the lead is fully inserted into the lead bore, e.g., by viewing whether a tip of the lead protrudes past a setscrew block of the device.

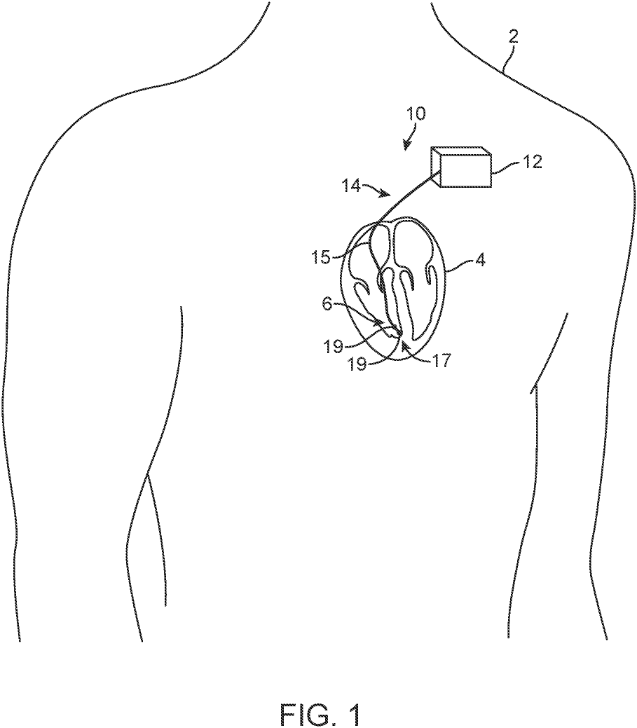

FIG. 1 is a schematic view of one embodiment of an implantable medical device system 10 disposed within a body of a patient 2. The system 10 includes an implantable medical device 12 and a lead 14 connected to the device. The system 10 can function as a single chamber, e.g., ventricular, pacemaker, as illustrated by the example of FIG. 1, or as a dual-chamber pacemaker that delivers pacing to a heart 4 of the patient 2.

As shown in the embodiment illustrated in FIG. 1, the lead 14 includes an elongated lead body 15 with a distal portion 17. The distal portion 17 is positioned at a target site 6 within the heart 4 of the patient 2. Distal portion 17 may include one or more electrodes 19. The target site 6 can be located at a wall of a ventricle of the heart 4. Further, the lead 14 can include any suitable lead, e.g., a bipolar or multipolar lead.

A clinician can maneuver distal portion 17 through the vasculature of the patient 2 to position the distal portion 17 at or near the target site 6. For example, the clinician may guide the distal portion 17 through the superior vena cava to the target site 6 on or in a ventricular wall of the heart 4, e.g., at the apex of the right ventricle as illustrated in FIG. 1. In one or more embodiments, other pathways or techniques may be used to guide the distal portions 17 into other target implant sites within the body of the patient 2. The system 10 may include a delivery catheter and/or outer member (not shown), and the lead 14 can be guided and/or maneuvered within a lumen of the delivery catheter to approach the target site 6.

The lead 14 can include one or more electrodes 19 adapted to be positioned on, within, or near cardiac tissue at or near the target site 6. In one or more embodiments, the electrodes 19 are adapted to provide pacing to the heart 4. The electrodes 19 can be electrically connected to conductors (not shown) extending through the lead body 15. In one or more embodiments, the conductors are electrically connected to circuitry such as therapy delivery circuitry of the implantable medical device 12, with the therapy delivery circuitry adapted to provide electrical signals through the conductor to electrodes 19. The electrodes 19 can conduct the electrical signals to the target tissue of heart 4, causing the cardiac muscle, e.g., of the ventricles, to depolarize and, in turn, contract at a regular interval. The electrodes 19 can also be connected to sensing circuitry of IMD 12 via the conductors, and the sensing circuitry may sense activity of the heart 4 via the electrodes 19. Such electrodes 19 may have various shapes such as tines, helices, screws, rings, and so on. Again, although a bipolar configuration of lead 14 including two electrodes 19 is illustrated in FIG. 1, in other embodiments IMD 12 can be coupled to leads including different numbers of electrodes, such as one electrode, three electrodes, or four electrodes.

In one or more embodiments, IMD 12 includes an electronics module contained within a housing, where the electronics module includes circuitry that may be adapted to deliver cardiac pacing. In the example of FIG. 1, the circuitry disposed within IMD 12 can include therapy delivery circuitry electrically coupled to electrodes 19. The circuitry within IMD 12 can also include sensing circuitry configured to sense electrical activity of heart 4 via electrodes 19. The therapy delivery circuitry can be configured to administer cardiac pacing via the electrodes 19, e.g., by delivering pacing pulses in response to expiration of a timer and/or in response to detection of the activity (or absence thereof) of heart.

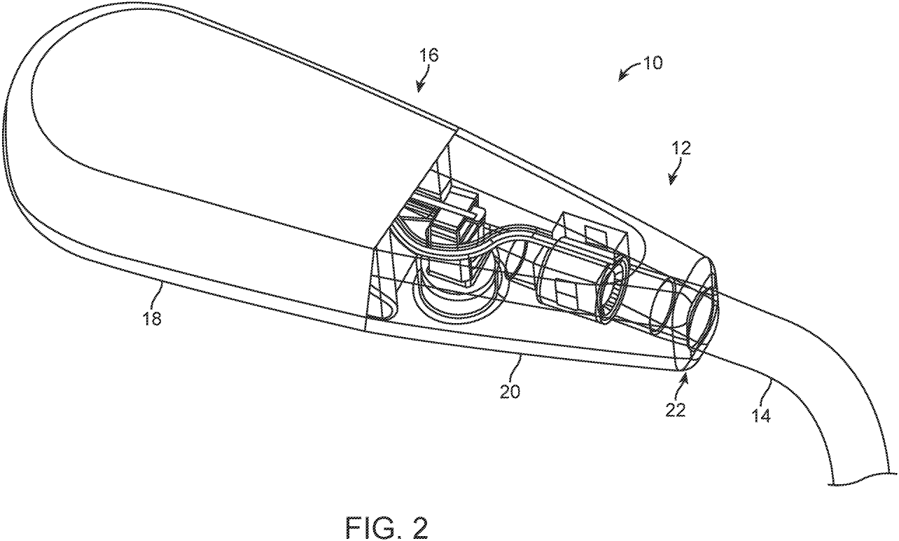

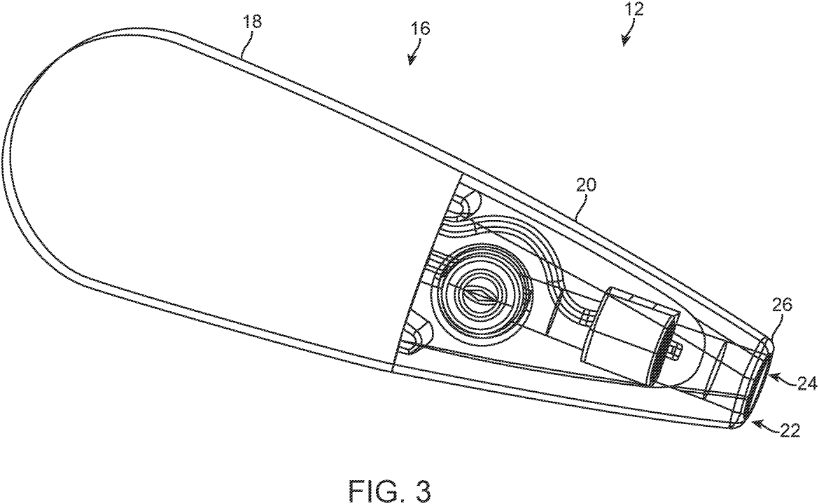

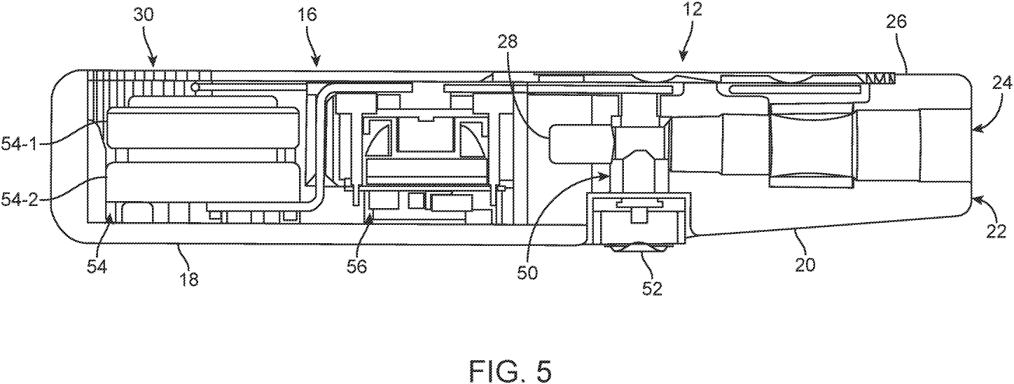

FIGS. 2-6 are various views of one embodiment of the implantable medical device system 10 of FIG. 1. The system 10 includes the implantable medical device (IMD) 12 and the lead 14. IMD 12 includes a housing 16 having a first portion 18 and a second portion 20 connected to the first portion. The housing 16 also includes a lead to bore 22 that extends between a first end 24 at an outer surface 26 of the housing and a second end 28 disposed within the housing (FIG. 5).

The IMD 12 of the system 10 can include any suitable medical device that is adapted to be implanted within a body of a patient. In one or more embodiments, the device 12 can be a pacemaker. Further, in one or more embodiments, the device 12 can be a leadless cardiac monitor. The device 12 can include any other suitable medical devices such as at least one of a pulse generator, defibrillator, insertable cardiac sensor, LVAD, neurostimulator, drug pump, glucose sensor, optical imaging device, or pressure sensor.

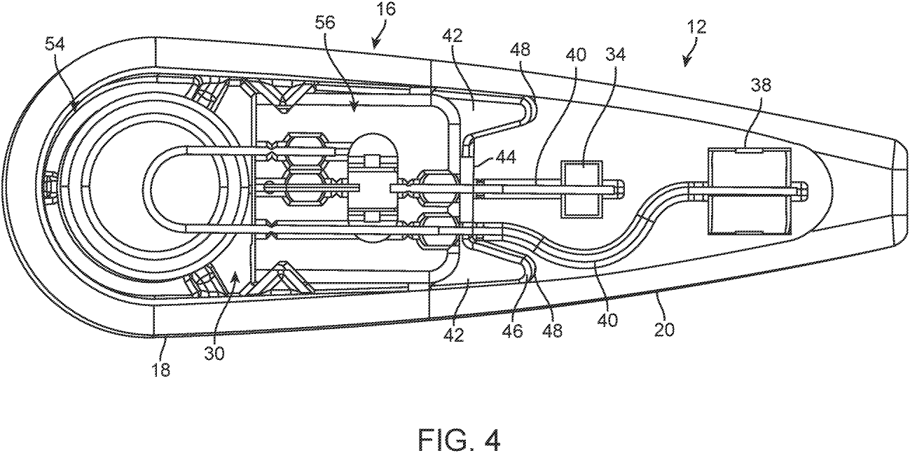

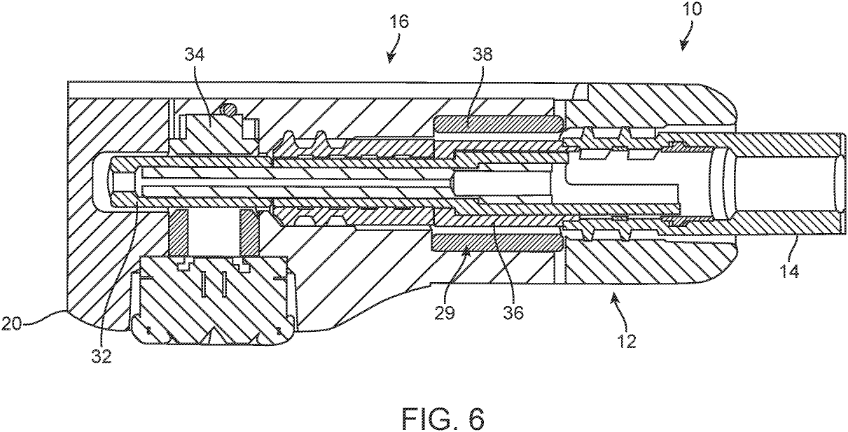

The system 10 also includes the lead 14 that is adapted to be electrically connected to an electronics module 30. The lead 14 can include any suitable lead, e.g., a cardiac pacing lead, defibrillation lead, left heart lead, epicardial leads, etc. Although depicted as including one lead 14, the system 10 can include any suitable number of leads that are electrically connected to the electronics module 30. The lead 14 can be electrically connected to the electronics module 30 that is disposed within the housing 16 using any suitable technique or techniques. In one or more embodiments, at least a portion 29 of the lead 14 can be disposed within the lead bore 22 of the housing 16 such that a lead contact 32 is electrically connected to contact 34 disposed within the lead bore when the at least a portion of the lead is disposed within the lead bore (FIG. 6). In one or more embodiments, the lead 14 can include a second lead contact 36 that is adapted to electrically connect the lead 14 to a second contact 38 disposed within the lead bore 22 when the portion 29 of the lead is disposed within the lead bore. The contact 34 and the second contact 38 can be electrically connected to an electronics module 30 by conductors 40 as is shown in FIG. 4.

The housing 16 of the device 12 can take any suitable shape or shapes and have any suitable dimensions. Further, the housing 16 can include any suitable material or materials, e.g., at least one of a metallic, polymeric, or inorganic material. Suitable materials for the housing 16 can include at least one of titanium (e.g., any suitable grade such as grade 5 titanium), stainless steel, polymer, ceramic, glass, or combinations thereof such as laminates, composites, or miscible blends or mixtures. In one or more embodiments, the housing 16 can include any suitable polymeric material or materials, e.g., at least one of epoxy, polyurethane, silicone, polyolefin, acrylic polymer, polyester, polyethelether ketone, liquid crystal polymer, polysulfone, polymethylene oxide, or polyvinyl, or combinations thereof.

The housing 16 can be a unitary housing. In one or more embodiments, the housing 16 can include two or more portions that are connected using any suitable technique or techniques, e.g., welding, mechanically fastening, adhering, thermal bonding, diffusion bonding, laser-assisted diffusion bonding, solvent bonding, over-molding, etc. For example, the housing 16 can include the first portion 18 and the second portion 20. The first portion 18 can include the same material or materials as those of the second portion 20. In one or more embodiments, the first portion 18 includes a material that is different from a material of the second portion 20.

In one or more embodiments, the first portion 18 of the housing 16 includes a polymer material. In one or more embodiments, the polymer material can include at least one of a thermoplastic polymer material, a thermoset polymer material, a polymer composite, or a polymer blend. Suitable polymer materials include, e.g., liquid-crystal polymer (LCP), polyetherether ketone (PEEK), polyether kotone (PEK), polyoxidemethylene (POM), polyimide, polypropylene, polyurea, polyester, polyphenylene oxide, polyamide, polytetrafluorethylene, polysulfone, polycarbonate, polystyrene or its copolymer, polyacrylate, polymethylacrylate, cellulose derivative, epoxy, or phenolic resin. The polymer material of the first portion 18 of the housing 16 can have any suitable crystallinity, e.g., crystallinity of at least 1%. Further, the polymer material of the first portion 18 can include a T_{M} of at least 50° C. In addition, the polymer material can include a water solubility, e.g., of no greater than 5%. Although depicted as being opaque, the first portion 18 of the housing 16 can instead be transparent or translucent.

Further, in one or more embodiments, the second portion 20 of the housing 16 includes a transparent polymer material. Such transparent polymer material can include at least one of polyurethane, epoxy, silicone, acrylic, polyolefin, polystyrene, polyimide, and copolymers, etc. As used herein, the term “transparent” means that that at least a portion of the second portion 20 of the housing 16 has a transmission value of at least 50 % for visible light. In general, the second portion 20 of the housing 16 can be transparent such that a clinician or technician can more easily guide at least a portion 29 of the lead 14 into the lead bore 22 and visibly confirm that contacts of the lead are in contact with contacts of the device 12.

The first and second portions 18, 20 of the housing 16 can be formed using any suitable technique or techniques, e.g., molding, thermoforming, laminating, over-molding, casting, insert molding, etc. In one or more embodiments, the first and second portions 18, 20 are formed separately and then connected together using any suitable technique or techniques. In one or more embodiments, the first portion 18 of the housing 16 is molded to the second portion 20 of the housing using any suitable molding technique or techniques. In one or more embodiments, the first portion 18 is adhered to the second portion 20 using any suitable adhesive or adhesives. Further, in one or more embodiments, the first portion 18 is mechanically fastened to the second portion 20 using any suitable technique or techniques, e.g., one or screws or other fixation devices.

In one or more embodiments, the housing 16 can include at least one tab 42 disposed on a mating surface 44 of the first portion 18 of the housing, where the at least one tab is adapted to receive respective tabs or slots 46 disposed on a mating surface 48 of the second portion 20 of the housing as is shown in FIG. 4. As illustrated, first portion 18 includes two tabs 42 disposed on mating surface 44 that are adapted to engage with slots 46 that are disposed on the mating surface 48 of the second portion 20 of the housing. Such tabs and slots 42, 46 are adapted to connect the first portion 18 to the second portion 20 and keep such portions mated together. In one or more embodiments, an adhesive can be disposed between the tabs 42 and slots 46 such that the first portion 18 remains connected to the second portion 20. In one or more embodiments, the first portion 18 can be molded or overmolded to the second portion 20 such that the tabs 42 and slots 46 act as molding features that aid the molding process in connecting the first and second portions together.

In one or more embodiments, the first portion 18 of the housing 16 can enclose the second portion 20 of the housing, i.e., the second portion can be circumscribed or enclosed by the first portion. In one or more embodiments, the second portion 20 forms a window or windows within the first portion 18 of the housing 16.

The housing 16 can include any suitable ports or receptacles that can connect the device to external components or systems. For example, the housing 16 can include the lead bore 22 disposed in any suitable portion or portions of the housing. In one or more embodiments, the lead bore 22 is disposed in the second portion 20 of the housing. As shown in FIGS. 5-6, the lead bore 22 extends between the first end 24 and the second end 28. The first end 24 of the lead bore 22 can be disposed at the outer surface 26 of the second portion 20 of the housing 16 and the second end can be disposed within the housing. The lead bore 22 can take any suitable shape or shapes and have any suitable dimensions. The housing 16 can also include a setscrew bore 50 disposed in any suitable portion or portions of the housing that is adapted to receive a setscrew block 52. The setscrew bore 50 can take any suitable shape or shapes and have any suitable dimensions. The setscrew block 52 can include any suitable fastener that is adapted to retain the lead 14 in the lead bore 22.

Disposed within the housing 16 is the electronics module 30, which can include any suitable electronic components or circuitry. In one or more embodiments, the module 30 can include a power source 54 and circuitry 56 that is electrically connected to the power source. Although not shown, in one or more embodiments, at least one of the power source 54 or the circuitry 56 can be disposed on a substrate or substrates.

The power source 54 can include any suitable power source or sources, e.g., one or more batteries. As shown in FIGS. 4-5, the power source 54 includes a first power source 54-1 and a second power source 54-2 (collectively power source 54). Although depicted as including two power sources, the power source 54 can include any suitable number of power sources, e.g., one, two, three, four, five, or more power sources. The power source 54 can be disposed in the housing 16 in any suitable location.

Electrically connected to the power source 54 is circuitry 56, which can include any suitable device or component, e.g., at least one of a capacitor, transistor, integrated circuit including a controller or multiplexer, sensor, accelerometer, inductive charging coil, antenna, optical components such as emitters and detectors, etc. The components can be electrically connected to the power source 54 or one or more additional components using any suitable techniques or techniques. Further, the circuitry 56 can be disposed within or on the housing 16 and electrically connected to the power source 54.

In one or more embodiments, a coating or coatings can be disposed over at least a portion of the outer surface 26 of the housing 16 using any suitable technique. The external coating can include any suitable material or materials, e.g., a biocompatible material. Suitable biocompatible coatings can include at least one of a metallic, ceramic, or polymeric material. Suitable metallic materials include a sputter metallic coating or foil. Further, suitable ceramic materials include at least one of aluminum oxide, glass, mica, titanium oxide, titanium nitride, vanadium oxide, niobium oxide, zirconium oxide, hafnium oxide, silicon oxide, or silicon nitride. And suitable polymer materials include at least one of parylene, silicone, acrylic, or epoxy. In one or more embodiments, the coating can include a multilayer coating having any suitable material or materials, e.g., at least one of epoxy, parylene, polyimide, silicone, acrylic, or vinyl.

The implantable medical device 12 can further include any suitable materials disposed within the housing 16. For example, a desiccant can be disposed in any suitable location within the housing 16. The desiccant can include any suitable material or materials that can absorb moisture present within the housing 16, e.g., at least one of a molecular sieve, calcium sulfate, silica gel, lithium sulfate, or polyanhydride.

Further, one or more polymeric fillers can be disposed in any suitable location within the housing 16. The polymeric fillers can include any suitable material or materials, e.g., at least one of a medical adhesive, a medical grade silicone, an epoxy, ethylene vinyl acetate (EVA), acrylic, glass fiber, carbon fiber, colorants like Ti02, or barium sulfate.

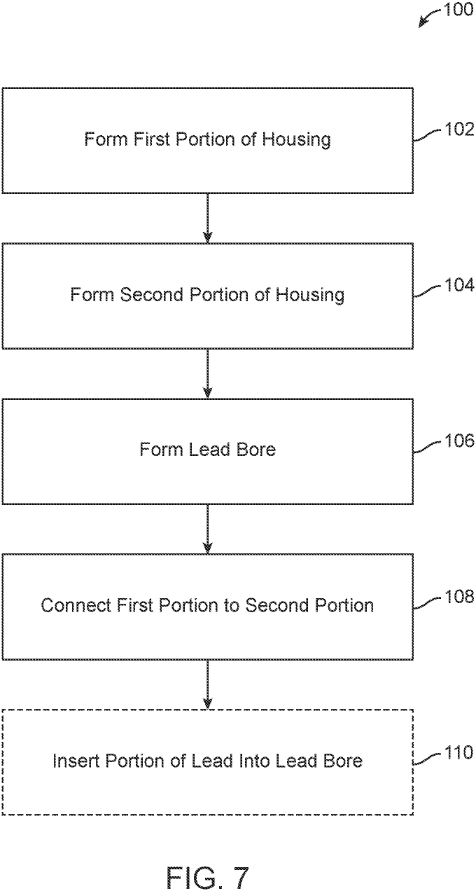

Any suitable technique or techniques can be utilized to form the implantable medical device system 10 of FIG. 1 and the implantable medical device 12 of FIGS. 2-6. For example, FIG. 7 is a flowchart of one method 100 of forming the implantable medical device system 10. Although described in reference to medical device system 10 of FIG. 1 and an implantable medical device 12 in FIGS. 2-6, the method 100 can be utilized to form any suitable implantable medical device system that includes an implantable medical device. At 102, the first portion 18 of the housing 16 can be formed using any suitable technique or techniques. In one or more embodiments, the first portion can include at least one of a thermoplastic polymer material or a thermoset polymer material. The second portion 20 of the housing 16 can be formed at 104 using any suitable technique or techniques. Such second portion 20 can include, e.g., a transparent polymer material. At 106, the lead bore 22 can be formed in the second portion 20 of the housing 16 using any suitable technique such that the lead bore extends between the first end 24 at the outer surface 26 of the housing and the second end 28 disposed within the housing. Further, the method 100 includes connecting the first portion 18 of the housing 16 to the second portion 20 of the housing at 108 using any suitable technique. For example, the second portion 20 of the housing 16 can be molded onto the first portion 18, overmolded onto the first portion, etc. Further, in one or more embodiments, the at least one tab 42 can be formed on the mating surface 44 of the first portion 18 and connected to the at least one slot 46 formed on the mating surface 48 of the second portion 20 using any suitable technique. The at least one tab 42 of the first portion 18 is adapted to mate with the respective at least one slot 46 of the second portion 20 when the first portion is connected to the second portion.

At 110, the at least a portion 29 of the lead 14 can be inserted into the lead bore 22 such that one or more of the lead contacts (e.g., lead contacts 34, 38 of FIG. 6) of the lead is electrically connected to the circuitry 56 of the electronics module 30 disposed within the housing 16 of the implantable medical device 12.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An implantable medical device comprising a housing that comprises a first portion and a second portion connected to the first portion, the first portion comprising a polymer material and the second portion comprising a transparent polymer material, wherein the housing further comprises a lead bore that extends between a first end at an outer surface of the housing and a second end disposed within the housing.

2. The device of claim 1, wherein the polymer material of the first portion of the housing comprises at least one of a thermoplastic material, a thermoset material, a polymer composite, or a polymer blend.

3. The device of claim 2, wherein the polymer material of the first portion of the housing comprises a thermoplastic polymer material comprising at least one of liquid-crystal polymer (LCP), polyetherether ketone (PEEK), polyether kotone (PEK), polyoxidemethylene (POM), polyimide, polypropylene, polyurea, polyester, polyphenylene oxide, polyamide, polytetrafluorethylene, polysulfone, polycarbonate, polystyrene or its copolymer, polyacrylate, polymethylacrylate, cellulose derivative, epoxy, or phenolic resin.

4. The device of claim 1, wherein the polymer material of the first portion of the housing comprises a crystallinity of at least 1%.

5. The device of claim 1, wherein the polymer material of the first portion of the housing comprises a Tₘ of at least 50° C.

6. The device of claim 1, wherein the polymer material of the first portion of the housing comprises a water solubility of no greater than 5%.

7. The device of claim 1, wherein the transparent polymer material comprises polyurethane.

8. The device of claim 1, further comprising an electronics module disposed within the housing and comprising a power source and circuitry electrically connected to the power source.

9. The device of claim 8, wherein the lead bore is adapted to connect an external lead to the circuitry of the electronics module.

10. An implantable medical device system comprising an implantable medical device and a lead, wherein the implantable medical device comprises:
a housing comprising a first portion and a second portion connected to the first portion, the first portion comprising a polymer material and the second portion comprising a transparent polymer material, wherein the housing further comprises a lead bore that extends between a first end at an outer surface of the housing and a second end disposed within the housing; and
an electronics module disposed in the housing and comprising a power source and circuitry electrically connected to the power source;
wherein at least a portion of the lead is adapted to be disposed within the lead bore and electrically connected to the circuitry of the electronics module.

11. The system of claim 10, wherein the lead bore of the implantable medical device comprises a contact that is electrically connected to the circuitry of the electronics module by a conductor, wherein a lead contact of the lead is adapted to be electrically connected to the contact of the lead bore when the at least a portion of the lead is disposed within the lead bore.

12. The system of claim 10, wherein the polymer material of the first portion of the housing of the implantable medical device comprises at least one of a thermoplastic polymer material, a thermoset polymer material, a polymer composite, or a polymer blend.

13. The system of claim 12, wherein the polymer material of the first portion of the housing of the implantable medical device comprises at least one of PEEK or LCP.

14. The system of claim 10, wherein the transparent polymer material of the housing of the implantable medical device comprises polyurethane.

15. The system of claim 10, further comprising a ceramic coating disposed over at least a portion of an outer surface of the housing of the implantable medical device.

16. The system of claim 15, wherein the ceramic coating comprises at least one of aluminum oxide, glass, mica, titanium oxide, titanium nitride, vanadium oxide, niobium oxide, zirconium oxide, hafnium oxide, silicon oxide, or silicon nitride.

17. The system of claim 10, further comprising a multilayer coating disposed over at least a portion of an outer surface of the housing, wherein the multilayer coating comprises at least one of epoxy, parylene, polyimide, silicone, acrylic, or vinyl.

18. A method of forming an implantable medical device system, comprising:
forming a first portion of a housing comprising a polymer material;
forming a second portion of the housing comprising a transparent polymer material;
forming a lead bore in the second portion of the housing that extends between a first end at an outer surface of the housing and a second end disposed within the housing; and
connecting the first portion of the housing to the second portion of the housing.

19. The method of claim 18, wherein connecting the first portion of the housing to the second portion of the housing comprises molding the second portion onto the first portion.

20. The method of claim 19, wherein molding the second portion of the housing onto the first portion of the housing comprises overmolding the second portion onto the first portion.
